(19) United States
(12) Patent Application Publication
(10) Pub. No.: US 2011/0313894 A1
(43) Pub. Date: Dec. 22, 2011

(54) System and Method for Surgical Pack Manufacture, Monitoring, and Tracking

(76) Inventors: Alan W. Dye, Mundelein, IL (US); Michael S. McMcMahon, Libertyville, IL (US)

(21) Appl. No.: 13/155,200

(22) Filed: Jun. 7, 2011

Related U.S. Application Data
(60) Provisional application No. 61/356,113, filed on Jun. 18, 2010.

(51) Int. Cl. G06Q 10/00; G06Q 30/00; B65B 11/00; G06K 7/01

(52) U.S. Cl. 705/28; 340/10.1; 705/34; 53/461

(57) ABSTRACT

A wrapped surgical pack (400) includes a plurality of adhesive bindings (301,302) that retain an identification device (305) against an outer wrap (201) of the wrapped surgical pack (400). The identification device (305) is capable of being read by a remote reader (801). When the wrapped surgical pack (400) is opened, the physical structure of the identification device (305) is altered, thereby allowing one or more processors operable with the remote reader (801) to detect the opening event. A system (800) using the identification device (305) can be used for order fulfillment, inventory management, location tracking, and other systems.

### BACKGROUND

1. Technical Field

This invention relates generally to the manufacture, monitoring, and tracking of surgical packs, and more particularly to a methods and systems of making and using custom surgical packs having radio frequency identification (RFID) tags capable of detecting use of the surgical packs to coordinate manufacturing and inventory control.

2. Background Art

Manufacturers such as Medline Industries of Mundelein, Ill., manufacture customized surgical packs for medical service providers such as doctors, nurses, and surgeons. These surgical packs, which are also known as “sterile procedure trays” or SPTs, and are alternatively known as “surgical kits,” contain all implements for a particular surgical procedure. The implements are generally disposable, and include supplies such as scalpels, drapes, and bandages, and so forth. Illustrating by way of example, an appendectomy surgical pack for Hospital ABC may include twenty or thirty implements specified by the hospital for performing that procedure. When an appendectomy is scheduled, one of the appendectomy specific surgical packs is retrieved from the stock room and is delivered to the operating room. The surgeon and nurses use each of the items during the procedure and then dispose of the implements after use.

While surgical packs for routine procedures may include only twenty to thirty elements, some surgical packs can include as many as two hundred implements. Most all surgical packs are customized, and are packed to each customer's specific requirements. Different customers may require different components for each procedure. For example, one hospital may suture a wound with degradable suture material, while another may prefer non-degradable suture material, and so forth. Some manufacturers offer tens of thousands of different types of surgical packs.

One problem associated with these surgical packs involves inventory management. Given the large number of medical procedures that can be performed, a medical service provider must keep a large variety of surgical packs on hand. To preserve storage space, most medical service providers prefer to keep only a few of each type of pack in inventory. For instance, a particular hospital may only have three or four appendectomy packs, three or four heart surgery packs, and so forth. If a medical service provider were to run out of a particular pack, problems can arise. For example, when a patient needs an appendectomy, they generally need it right away. If the hospital or surgeon was without appendectomy packs, health risks may increase for the patient due to delays in having the appendix removed.

There is thus a need for an inventory and tracking system for surgical packs.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates one surgical pack in accordance with embodiments of the invention.

FIG. 2 illustrates one surgical pack being wrapped in accordance with embodiments of the invention.

FIG. 3 illustrates one wrapped surgical pack configured in accordance with embodiments of the invention.

FIG. 4 illustrates one wrapped, sealed surgical pack configured in accordance with embodiments of the invention.

FIG. 5 illustrates one tag configured in accordance with embodiments of the invention.

FIG. 6 illustrates another tag configured in accordance with embodiments of the invention.

FIG. 7 illustrates another tag configured in accordance with embodiments of the invention.

FIG. 8 illustrates a system configured in accordance with embodiments of the invention.

FIG. 9 illustrates a method configured in accordance with embodiments of the invention.

FIG. 10 illustrates a method configured in accordance with embodiments of the invention.

FIG. 11 illustrates a method configured in accordance with embodiments of the invention.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION OF THE INVENTION

Before describing in detail embodiments that are in accordance with the present invention, it should be observed that the embodiments reside primarily in combinations of method steps and apparatus components related to ordering, tracking, manufacturing, and inventory management of surgical packs. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

It will be appreciated that embodiments of the invention described herein may be comprised of one or more conventional processors and unique stored program instructions that control the one or more processors to implement, in conjunction with certain non-processor circuits, some, most, or all of the functions of tracking, manufacturing, provisioning, and inventory management of surgical packs as described herein. The non-processor circuits may include, but are not limited to, a radio receiver, a radio transmitter, signal drivers, clock circuits, power source circuits, and user input devices. As such, these functions may be interpreted as steps of a method to perform the tracking, manufacturing, provisioning, and inventory management steps associated with surgical packs as described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ASICs with minimal experimentation.

Embodiments of the invention are now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, and the like may be used solely to distinguish one entity or action from another entity or action without necessarily requiring or implying any actual such relationship or order between such entities or actions. Also, reference designators shown herein in parenthesis indicate components shown in a figure other than the one in discussion. For example, talking about a device (10) while discussing figure A would refer to an element, 10, shown in figure other than figure A.

Managing inventory with respect to customized and specialized surgical packs is a very difficult task. For example, manual counting procedures cannot always be relied upon. Most all surgical packs include some sterilized components. As will be described below, these packs can be shipped with a wrap disposed about the packs to keep the contents within sterile. Where the wrap has been compromised, for example where some unwrapping has occurred due to inadvertent contact, an inventory manager may count the pack as being available while it is in fact not available, due to the sterility having been compromised.

To further complicate matters, most hospitals and other medical service providers do not have automated ways to track the inventory of surgical packs as they are moved from stock room to operating room and so forth. Further, most hospitals and ambulatory surgical centers do not have the staff to appropriately take inventory of the different packs on hand. As noted above, manufacturers can produce thousands of such packs, each with different contents and being intended for a different procedure. The time necessary to read labels on each pack—most of which look identical from the outside—can easily overwhelm most medical staffs. As a result, the probability of a shortage occurring is high. When a person needs a particular surgery, and the appropriate surgical pack is not in inventory, potentially life threatening consequences can result.

Due to their customized nature, manufacturing lead times associated with surgical packs can be quite long. This long lead time exacerbates the shortage problem mentioned in the preceding paragraph, as it may take six weeks to manufacture a lot of a particular surgical pack. Further, the lead time to book the manufacturing and shipping process can be as long as four weeks. As one might imagine, the need for a particular type of surgery is entirely case based, and is not consistent. To the contrary, the need to perform a particular operation or procedure will have peaks and valleys based upon the number of patients requiring the procedure.

Rather than maintaining an excessively high inventory of each surgical pack, as well as maintaining the excessive amount of storage required, embodiments of the present invention provide methods and apparatuses that can be used to manage inventory in multiple locations throughout a hospital or surgical center without the need of constant, manual inventory counts by staff. Embodiments of the present invention further work to identify when the surgical packs described herein have been opened for use. The determination of when the surgical pack is opened can be used to generate replacement orders. Accordingly, embodiments of the present invention provide systems and method steps to create a “pull” type inventory management system where replenishment is based upon use, as opposed to “push” type systems where demand forecasting is required.

Embodiments of the present invention employ Radio Frequency Identification (RFID) tags that are specialized in design so as to determine when a wrap material, such as a Central Sterile Reprocessing (CSR) wrap, is at least partially unfolded about a surgical pack. In one embodiment, the RFID tag includes two RFID tags, with at least one having a transmission antenna that spans adhesive bindings that hold the folded wrap layer together about the surgical pack. When opened, the transmission antenna is severed, thereby rendering one of the tags inoperable. Accordingly, a manufacturer can determine when the surgical pack has been used, and can accordingly requisite a new one to be made in the manufacturing process.

Surgical packs equipped with the identification devices can be monitored by one or more RFID interrogation devices that are disposed in various locations in the hospital or surgical. Information relating to use of the surgical packs can be fed back through a network to a server that is accessible by the manufacturer, so that the proper orders stemming from use can be created.

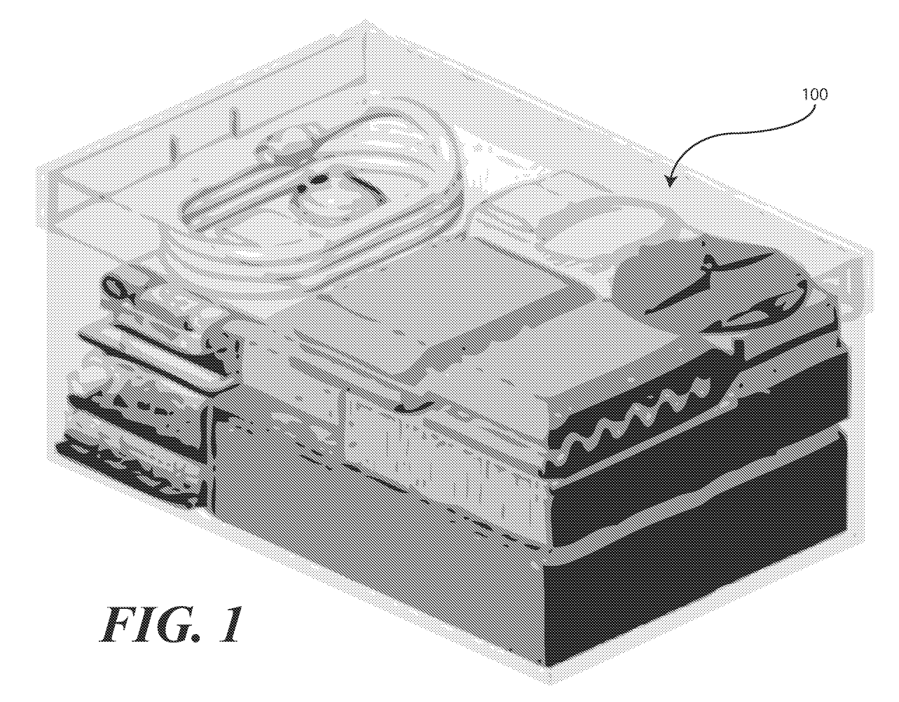

Turning now to FIG. 1, illustrated therein is one exemplary surgical pack 100 suitable for use with embodiments of the invention. In one embodiment, the surgical pack 100 is configured for disposable use, i.e., each item in the surgical pack 100 is intended to be used once during a procedure, then discarded. Most surgical packs 100 include between twenty and thirty items. However, some can include as many as 200 items.

In one embodiment, the surgical pack 100 is customized for both a particular procedure and a particular end user. As noted above, a surgical pack for an appendectomy procedure in one surgical center can include items that are different from a similar pack in another surgical center due to the preferences of the medical staff.

Examples of items that can be included in the surgical pack 100 include tubing, trays, bandaging materials, scalpels, syringes, needle and blade disposal devices, drapes, clamps, suturing implements, disinfectants, antibiotic creams and lotions, masks, gloves, and so forth. As each surgical pack is tied to a particular procedure, it will be clear to those of ordinary skill in the art having the benefit of this disclosure that other implements may be included with the surgical pack as well.

One or more of the components within the surgical pack 100 may be sterile. For example, in some embodiments some of the implements will be sterile, having undergone a sterilization process in manufacture, while others are non-sterile. In other embodiments, all of the implements in the surgical pack 100 may be sterile.

In practice, the sterilization process can include the following process when the surgical pack 100 is manufactured: pre-conditioning, sterilization, and post conditioning. This process, which varies from manufacturer to manufacturer can take between four to seven days to complete. One advantage of embodiments of the present invention is that the identification tags can be used not only to track usage and inventory of the surgical packs at the customer site, but at the manufacturing site as well. For instance, the identification tags configured in accordance with the present invention can equally be used to notify a prospective customer whether a pack is in the sterilization process, where it would be categorized as work in progress inventory, or whether the pack is ready for sale.

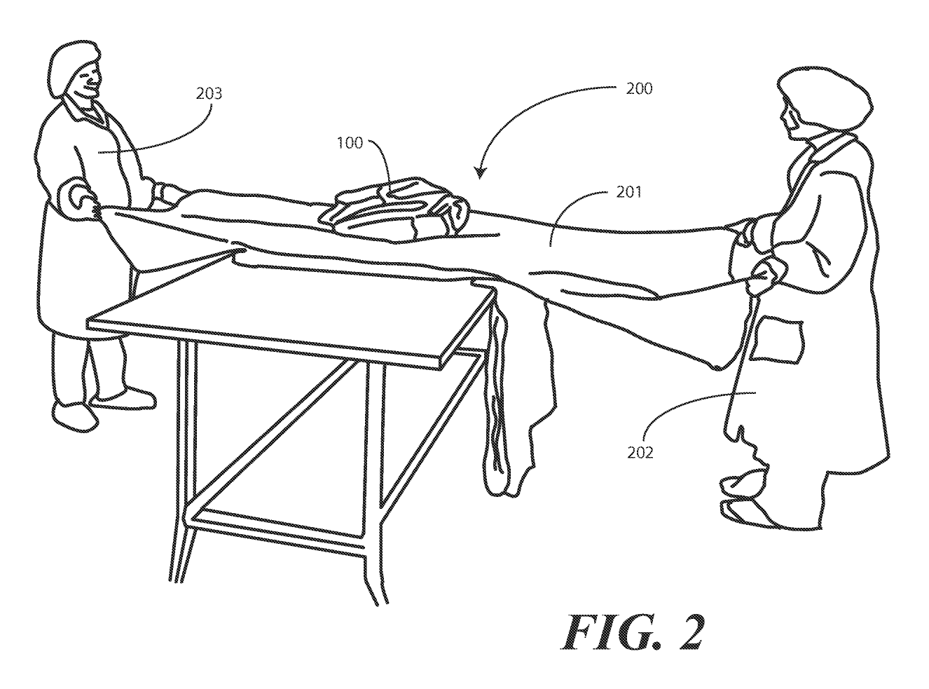

When the surgical pack 100 includes some sterile components and some non-sterile components, these components are paired together at a manufacturing step. Where the surgical pack 100 includes all sterile components, the pack will go directly from the sterilizing operation in manufacturing to inventory. In the latter case, wrapping occurs within the sterilization process. Turning now to FIG. 2, illustrated therein is one embodiment of the wrapping process.

Turning now to FIG. 2, illustrated therein is one step of the manufacturing process for surgical packs configured in accordance with embodiments of the invention. The step shown is the wrapping step 200.

Once the necessary components or implements are assembled to form the surgical pack 100, the surgical pack 100 can be sealed with a wrap 201 to keep the internal components sterile. The wrap 201 can be any of a variety of types of material. In one embodiment mentioned above, the wrap 201 is a CSR wrap. CSR wraps are widely used by medical professionals in hospitals, ambulatory surgical centers, and the like during medical procedures. While a CSR wrap is one example of a wrap that can be used, it will be clear to those of ordinary skill in the art that other wraps, such as plastic, cotton, linen, paper, or combinations thereof, can be substituted without departing from the spirit and scope of the invention.

Using a CSR wrap as an illustrative example, in one embodiment the CSR wrap 201 is folded about the surgical pack 100 for sealing and preventing unwanted debris from contaminating the surgical pack 100. The CSR wrap 201 can be correspondingly unfolded to open and reveal the surgical pack 100. In some embodiments, once unfolded, the CSR wrap 201 can then be used in the medical procedure. In simple examples, the CSR wrap 201 can be used to cover a table across which the contents of the surgical pack 100 can be spread. For instance, an unfolded CSR wrap 201 can be used to provide a sterile field atop which the contents of the surgical pack 100 can be placed for unloading and subsequent use.

As shown in FIG. 2, the surgical pack 100 is placed upon one or more layers of wrap material. The wrap material can take different sizes and shapes. In the illustrative embodiment of FIG. 2, the CSR wrap 201 is sufficiently large that the entire surgical pack 100 can be wrapped multiple times.

In the illustrative embodiment of FIG. 2, workers 202,203 are folding the CSR wrap 201 about the surgical pack 100 for illustration. It will be clear to those of ordinary skill in the art having the benefit of this disclosure that automated folding equipment could equally be used. Workers 202,203 are more conveniently illustrated, and so will be used to explain the folding process.

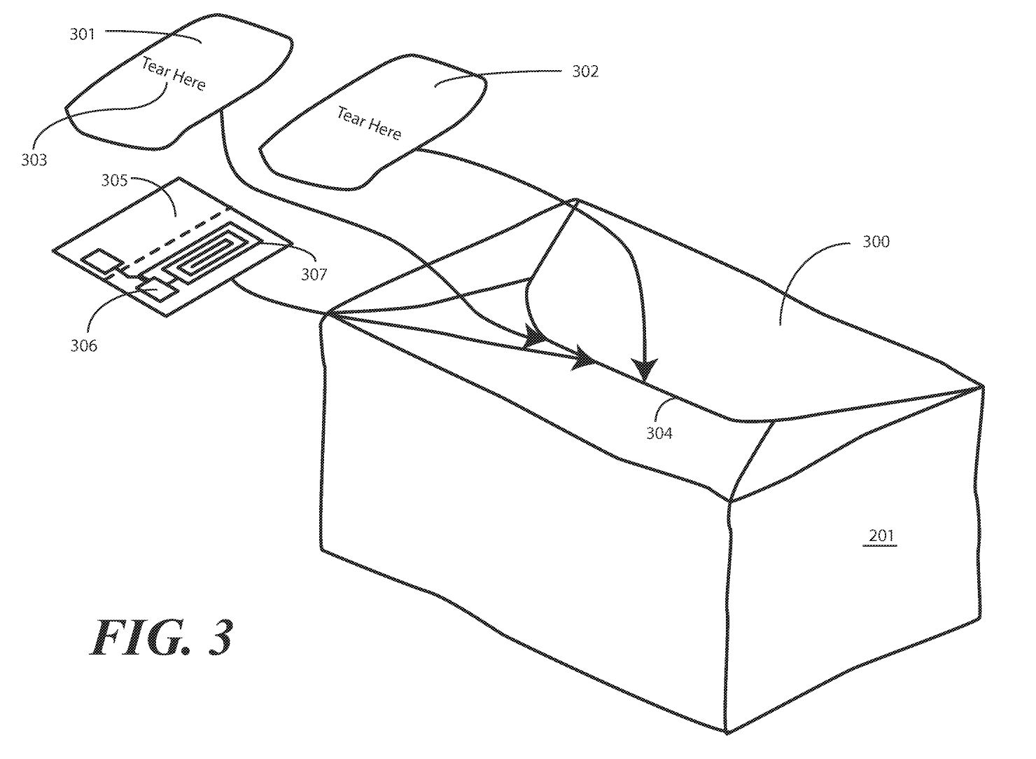

The workers 202,203 fold a first portion of the CSR wrap 201 about a first side of the surgical pack 100. The workers 202,203 then fold a second portion of the CSR wrap 201 about a second side of the surgical pack 100. A third portion of the CSR wrap 201 is then folded about a third side of the surgical pack 100. A fourth portion of the CSR wrap 201 is then folded about a fourth side of the surgical pack 100. The fourth portion, or any of the first portion, the second portion, the third portion, can be tucked beneath at least another of the first portion, the second portion, the third portion, or the fourth portion of the CSR wrap 201. The wrapped pack 300 is shown in FIG. 3.

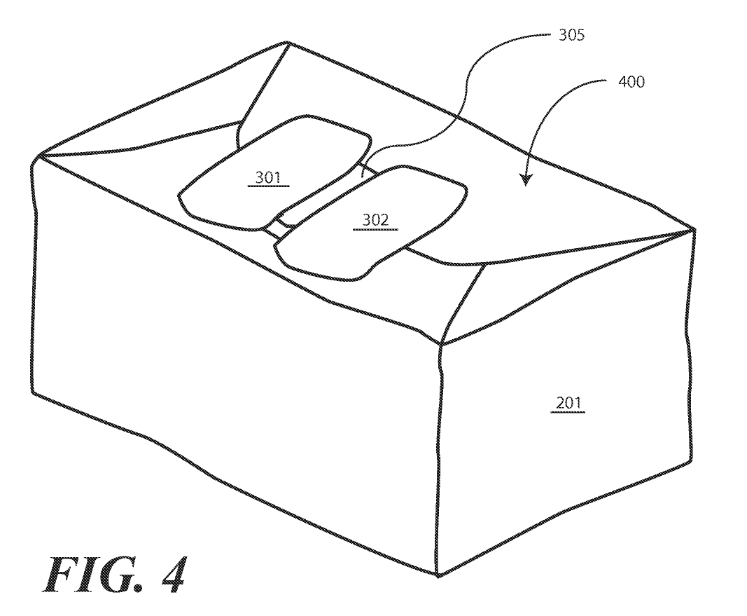

Once wrapped, in one embodiment of the invention the wrapped pack 300 is sealed with one or more adhesive bindings 301,302. The adhesive bindings 301,302 can take different forms, but in one embodiment comprise tearable labels having adhesive backing configured to stick to the CSR wrap 201. Note that while the embodiments of FIGS. 3 and 4 show two adhesive bindings 301,302 being used, it will be clear to those of ordinary skill in the art that embodiments of the invention are not so limited. For example, only one adhesive binding 301 can be used, or alternatively three or more adhesive bindings can be used as well.

In one embodiment, to assist the end user in knowing where to open the wrapped pack 300, each adhesive binding 301,302 includes an indication line 303. In one embodiment, the indication line reads “tear here.”

Disposed between the CSR wrap 201 and the adhesive bindings 301,302 is an identification device 305. The identification device 305 includes at least control device 306 and a radiating element 307. The control device 306 is capable of delivering all information necessary for identifying the surgical pack (100) to a remote reader, as will be described below.

In one embodiment, the identification device 305 is at least partially adhered to the adhesive bindings such that when one or both adhesive bindings 301,302 is torn across its indication line 303, at least a portion of the identification device 305 will be torn as well. For example, where one adhesive binding 301 is used, tearing that adhesive binding 301 will tear at least a portion of the identification device 305. Where two adhesive bindings 301,302 are used, they can be configured such that tearing one tears the identification device 305, or alternatively such that tearing both tears the identification device 305.

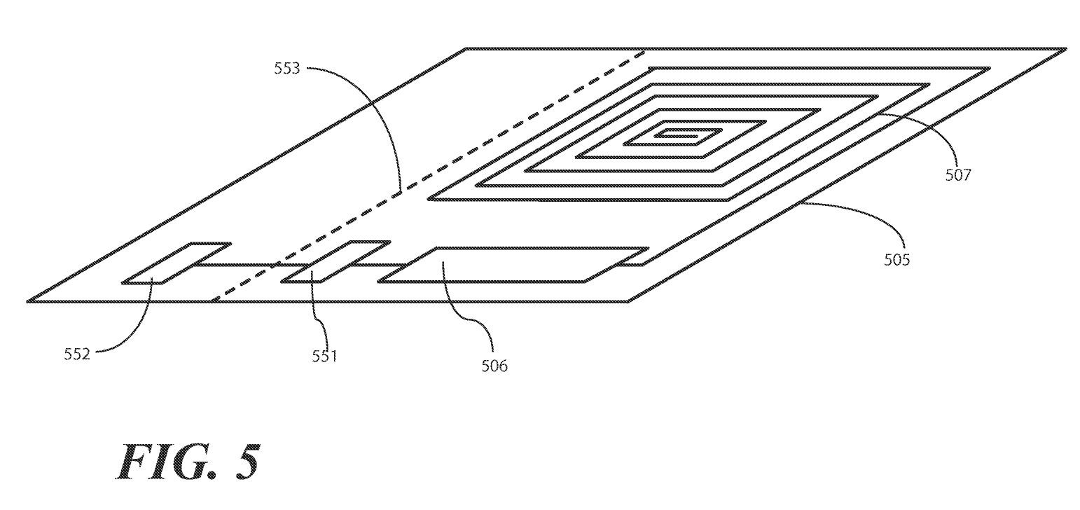

As will be described with respect to FIGS. 5-7, the tearing of the identification device 305 alters the information delivered by the control device 306 through the radiating element 307 to the remote reader, thereby letting the remote reader know that the wrapped pack 300 has been opened. The sealed pack 400, having the two adhesive bindings 301,302 coupled to the CSR wrap 201 and the identification device 305, thereby sealing the identification device 305 and the CSR wrap 201, is shown in FIG. 4.

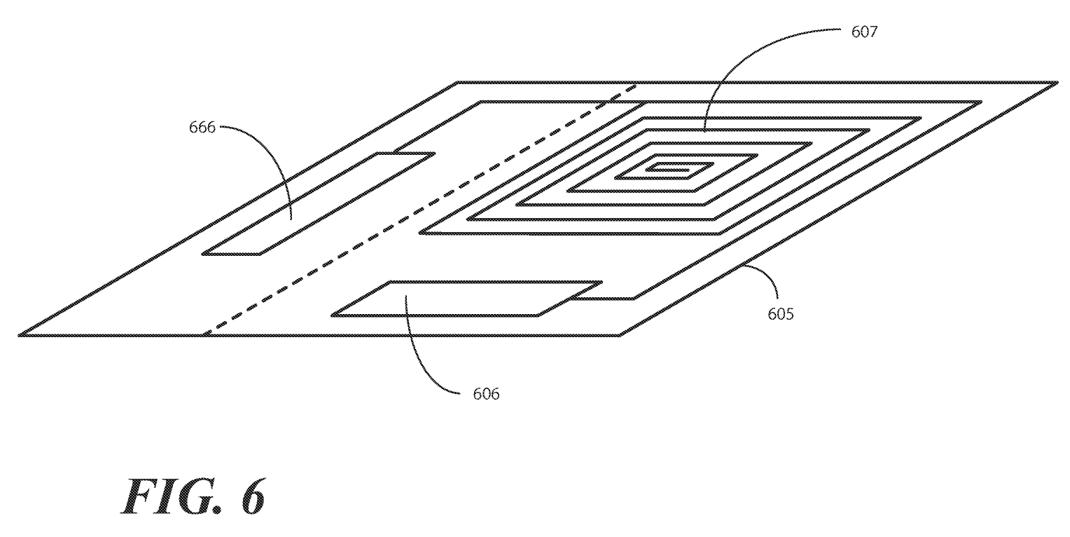

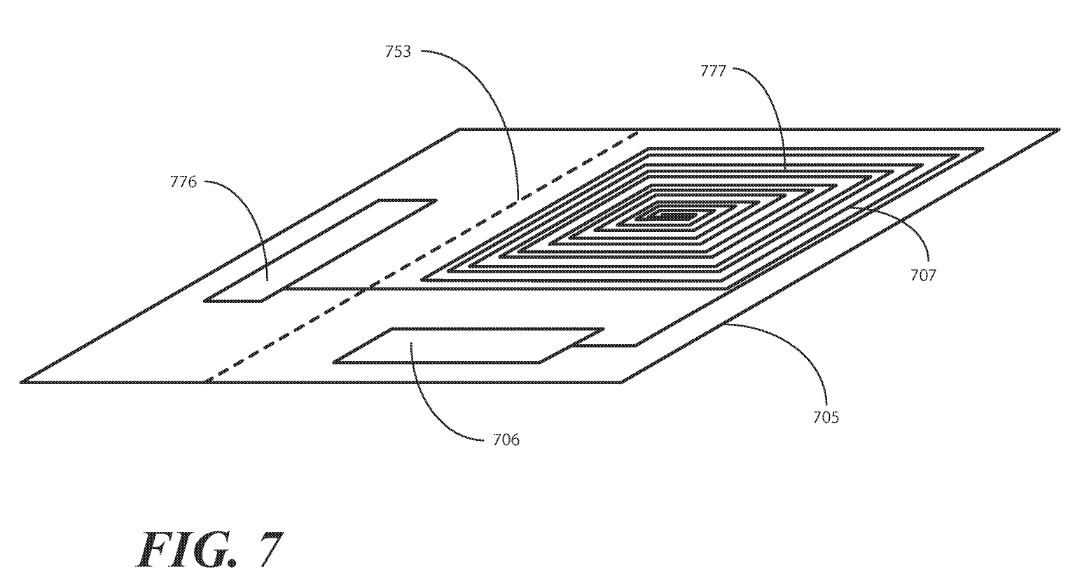

Turning now to FIGS. 5-7, illustrated therein are a few of the various identification devices 505,605,705 that can be used in accordance with embodiments of the invention. Each identification device 505,605,705 includes at least one control device 506,606,706 and at least one radiating element 507,607,707. The embodiment of FIG. 6 includes two control devices 606,666 sharing a common radiating element 607. The embodiment of FIG. 7 includes two control devices 706,776, each with its own radiating element 707,777.

Each identification device 505,605,705 can be configured as an RFID tag in accordance with RFID standards known in the art. For example, control devices 506,606,706,666,776 and corresponding radiating elements 507,607,707,777 can be configured as conventional passive or active RFID tag, similar in function to those available from manufacturers such as Fujitsu Limited and Philips Semiconductor.

Each of the control devices 506,606,706,666,776 is configured to be able to wirelessly identify its corresponding surgical pack (100) to a remote reader. For example, in one embodiment, the control devices 506,606,706,666,776 can comprise a memory and processor. Identification information concerning the contents of the surgical pack (100) can be stored in the memory. The memory can be read by the remote reader through the radiating elements 507,607,707,777. In addition to contents information, the memory can store other information as well, such as manufacture date, shipment date, expiration dates, customer information, and so forth. Other information can further include the inventory history of the article to which the identification devices 505,605,705 are attached. Accordingly, the date that the surgical pack (100) entered inventory, the date that the surgical pack (100) left inventory, whether the surgical pack (100) has been opened, any movement within inventory, and similar information.

The information stored in the memory may also include shipping manifests that indicate when and to whom the surgical pack (2010) is to be shipped. In one embodiment, information within the memory can be updated by the remote reader as well. For example, when a wrapped pack (300) is opened, the memory might be updated to indicate a replacement pack has been ordered. Each radiating element 507,607,707,777 is configured to function as a communication coil for near-field communication at a predetermined radio frequency, and communicates this information to the remote reader.

Determination of a wrapped pack (300) being opened can be determined in any of a variety of ways. Beginning with FIG. 5, the control device 506 is coupled to a first oscillation frequency determining component 551 and a second oscillation frequency component 552. These components can be impedance components used in conjunction with an RC timing circuit, or alternatively may be inductance components that work to determine a transmission frequency in an LC circuit. Alternatively, they can be capacitive devices that work to determine oscillation frequencies.

When the one or more adhesive bindings (301,302) are torn, the identification device 505 of FIG. 5 is configured to split along a perforation 553, thereby changing the oscillation frequency. By determining with which frequency the identification device 505 is communicating, the remote reader can determine whether the wrapped pack (300) has been opened.

Turning to FIG. 6, two control devices 606,666 share a common radiating element 607. When the two adhesive bindings (301,302) are torn, the identification device 605 of FIG. 6 is configured to split along a perforation 653, thereby disconnecting the second control device 666 from its radiating element 607. This renders the second control device 666 unable to communicate with the remote reader. By determining whether one or both of the control devices 606,666 is communicating, the remote reader can determine whether the wrapped pack (300) has been opened.

In one embodiment, one control device 606 is configured to provide an identifier to the remote reader. The identifier tells the remote reader to which pack the control device 606 is attached, and can include other information such as the contents of the pack, date of manufacturer, identity of the end user, and so forth. The other control device 666 is designed to present a second identifier, which is associated with the first identifier.

While the second identifier can be read by the remote reader, it is clear that the pack has not been opened. When the identification device 605 is torn along the perforation 653, the remote reader can no longer read the second identifier. Accordingly, the act of opening the pack can be determined.

Turning now to FIG. 7, while each control device 706,776 includes its own corresponding radiating element 707,777, the function is much the same as was in FIG. 6. As both radiating elements 707,777 are disposed on a common side of the perforation 753, tearing the perforation renders one control device 776 incapable of communicating with the remote reader. By determining whether one or both of the control devices 706,776 is communicating, the remote reader can determine whether the wrapped pack (300) has been opened.

The embodiments of FIGS. 5-7 illustrate but a few of the various options by which the physical structure of the identification devices 505,605,705 can be altered so as to be detected by a remote reader. Others will be obvious to those of ordinary skill in the art having the benefit of this disclosure.

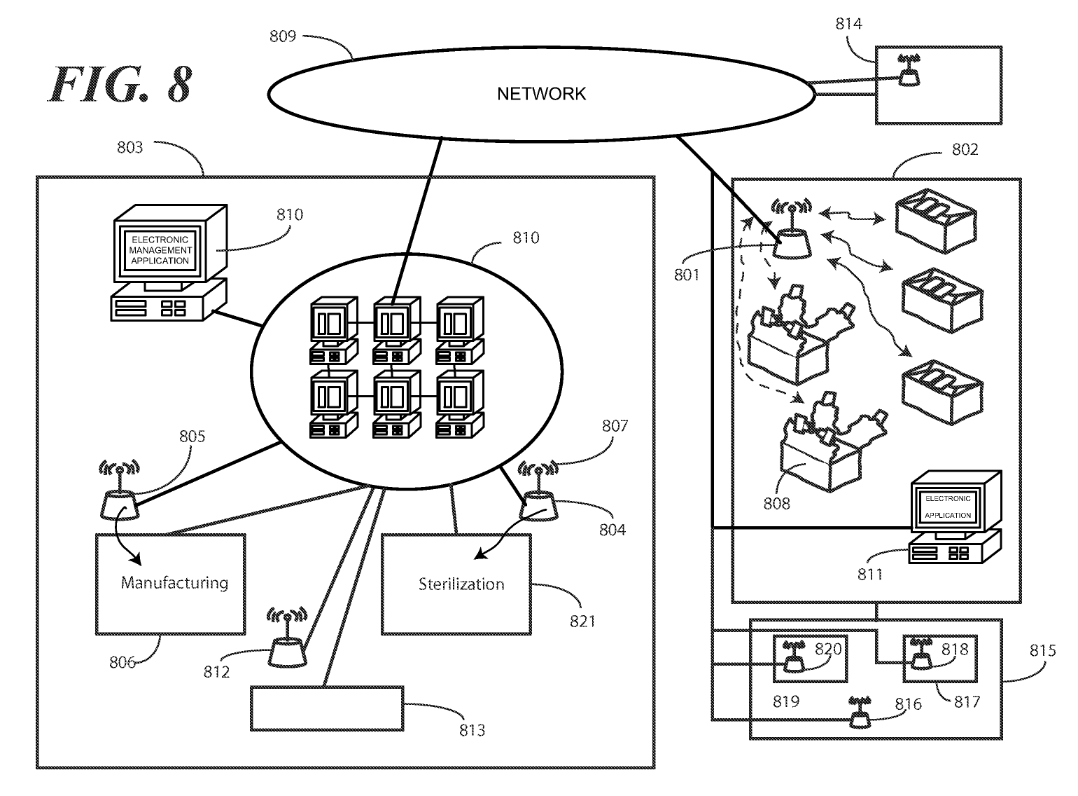

Turning now to FIG. 8, illustrated therein is a system for managing the distribution, use, and inventory management of surgical packs configured in accordance with embodiments of the invention. The embodiment of FIG. 8 can also be used to coordinate manufacturing and corresponding tracking as well.

In FIG. 8, one or more remote readers 801,804,805 are disposed in a location to be monitored. In one embodiment, the location 802 can be a hospital or surgery center. In another embodiment, the location 803 can be sites at the manufacturer, such as the manufacturing floor 806 or the sterilization area 821. Note that each of the remote readers 801,804,805 shown in FIG. 8 can represent a plurality of remote readers placed at various points within each location 802,803. In one embodiment, a sufficient amount of remote readers 801,804,805 is placed in each location such that all areas in which surgical packs may be are covered by the readers.

Each remote reader 801,804,805 communicates with the identification devices (305,505,605,705) by way of an electromagnetic field 807 emanating therefrom. Where the near-field communication is RFID communication, for example, this frequency may be about 13.56 MHz as is directed by recognized RFID standards. The remote readers 801,804,805 read the memories of each device to determine the information stored therein. Where a pack 808 has been opened, this can be determined by the remote reader 801 as described above.

Where the information is detected at the customer location 802, e.g., the hospital or surgical center, this information can be relayed through a network 809 to a server or computer system 810 at the manufacturer.

This information, retrieved from the end user, can serve a variety of purposes. These purposes can be for the benefit of the manufacturer, the end user, or combinations thereof. Illustrating by way of example, the information could be used by the manufacturer to track the location, status, and inventory of the surgical packs at the end user's location. Next, when an open pack 808 is detected, orders can be placed for replacement units with the manufacturer. Where the information is delivered to terminals 810,811 disposed at the manufacturer and end user locations 802,803, both the end user and the manufacturer can know when a new surgical pack is ordered, manufactured, or shipped in real time.

In one embodiment, the end user can, through its terminal 811, input instructions to have the manufacturer modify the contents of a particular surgical pack in future shipments as well. For example, perhaps a user determines that one component of the surgical pack is no longer necessary. That user can then remove the item by transmitting electronic instructions from the terminal 811 in its location 802 through the network 809 to the manufacturer's location 803. Similarly, the user may add an item or two through the network 809.

Customers can also detect, through instructions transmitted from their terminals 811, when the manufacturer is creating new inventory, as detected by its remote readers 804,805 tracking the packs in its manufacturing site. Further, the end user can determine whether the packs are on the manufacturing floor 806, are in the sterilization process (821), or are in the inventory holding area 813. Advantages of this system allow the end user to eliminate guesswork as to when a newly specified or ordered pack will be manufactured. It further reduces the need for the end user to call the manufacturer for status updates.

As noted above, in some assembly processes, some or all of each surgical pack goes through a sterilization step at the sterilization location 821. During this step, the surgical packs are placed within a chamber that is roughly the size of an eighteen-wheel truck trailer. Once the packs have been sterilized, they are transferred to the inventory holding area 813, where they sit for a predetermined time to allow sterilization gases to escape.

Using the system of FIG. 8, end users can determine, with the assistance of the manufacturer's readers 805,812, the network 809, and their terminal 811, the following: When each surgical pack in the sterilizer; Whether it is in sterilization process; or Whether it is in inventory allowing sterilization gas to escape. The system of FIG. 8 provides transparency to the end user for the sterilization schedule, and in so doing, works to prevent backorder situations.

After completing the sterilization step, as noted above some non-sterilized supplies can be combined with other sterilized components. When this occurs, the sterilized components leave the sterilization location 821 and go back to the manufacturing location 806 for pairing. In one embodiment of the invention, the identification devices, as sensed by the remote readers 805, are then coordinated with scheduling information in the manufacturer's computer system 810. This information can then be transmitted across the network 809 to the end user's terminal 811 so that the end user is aware that the surgical packs have left the sterilizer location 821 and have been delivered back to the manufacturing location 806 to be paired with non-sterile items.

Embodiments of FIG. 8 can also be used to provide the following functions:

1. To give the end user visibility at their terminal 811, based upon information delivered from the manufacturer system 810 across the network 809, to real-time data as to how many surgical packs are in warehouses 814 disposed locally with their location 802, as well as the schedule for delivering those packs to the end user's location 802.

2. To provide verification of inventory and location within the warehouse 814 to the manufacturer, so that the manufacturer knows how many units to build in a particular shipment.

3. In the unlikely event of a product recall, the system 800 of FIG. 8 can be used to attach hold identifiers with the pack identifiers stored in the manufacturer's system 810. For instance, in one embodiment an identifier can be attached to a product recall notice within a pack to be returned. The manufacturer can then notify the end user at their terminal 811 which packs need to be returned, and where the packs are stored at the end user's location 802.

4. As noted above, the manufacturer can provide real-time data for all surgical packs to the end user at all stages of the process.

5. If the end user has a remote reader 816 disposed at a receiving dock 815 at their location 802, either of the manufacturer or the end user can use the system 800 of FIG. 8 to check every single surgical pack on every single pallet as it is received. In a typical delivery process, between two and ten pallets of surgical packs are delivered at the receiving dock 815. With prior art systems, each box on each pallet had to be verified by either manually checking the item number printed on the box, or by manually scanning the barcode. Embodiments of the present invention dramatically reduce time spent receiving product at the receiving dock 815 by providing instant, cumulative scanning via the remote reader 816. The system 800 of FIG. 8 improves accuracy as well.

6. At the end user's location 802, other benefits of the system 800 become apparent. For example, once the boxes are received, they generally are transferred to a non-sterile storage area 817 just inside the receiving dock door. A remote reader 818 in this storage location can help the end user identify what stock is located therein. The system 800 can further allow for the inventory to be visible on the terminal 811, as well as assist in locating inadvertently misplaced packs.

7. After the initial storage in the non-sterile storage area 817, the packs are transferred to a sterile storage area 819. A supply sufficient for between two and five days is generally stored in this sterile storage area 819. A remote reader 820 disposed in this location can do the following: First, it can ensure that the end user is properly rotating its stock. Sterile surgical packs have expiration dates and need to be used on a FIFO basis. This system 800 helps ensure compliance with the FIFO usage. Second, the system 800 can help end users identify how long products are being stored.

8. If a component or implement has been added or is missing from a pack, a remote reader 801 within the end user's location 802 can detect this and highlight it in the end user's system 811. This can occur when a customized pack is ordered, or when the manufacturer needs to substitute a component due to a manufacturing or supply chain issue.

After storage in the sterile storage area 819, the surgical packs are generally placed on a cart for delivery to their end usage point, which is generally an operating room. This cart can sit in a hallway or other temporary storage location for a brief time. The remote readers 801 disposed at the end user's location 802 can determine when a particular surgical pack is pulled from the shelf. Further, the system 800 can determine when the surgical pack enters the operating room. Once the case cart is rolled into the surgical suite, the room is prepared for surgery. A remote reader in this location can determine when this occurs. Further, identification of the pack can be sensed by the remote reader and tied to the surgical schedule that is managed by the end user's computer system and is visible on the terminal 811. Accordingly, the system can use identification of the surgical pack to add a charge to the surgery recipient's bill.

Once the surgical pack is opened by removing the adhesive binders and tearing the identification device, it becomes an opened surgical pack 808. When this occurs, the opened surgical pack 808 must be used or discarded. It cannot be resealed, as the opening and resealing can compromise sterility. The inclusion of a remote reader capable of detecting when the pack is opened, in accordance with the description herein, can alert the manufacturer and end user to any of the following: First, the manufacturer can schedule production based on this event. Second, the end user can determine when each pack is used. Third, the manufacturer can offer a consignment program, where inventory is not purchased by the end user until opened. Such a system works to reduce end user inventory, as well as providing definable events that prove usage.

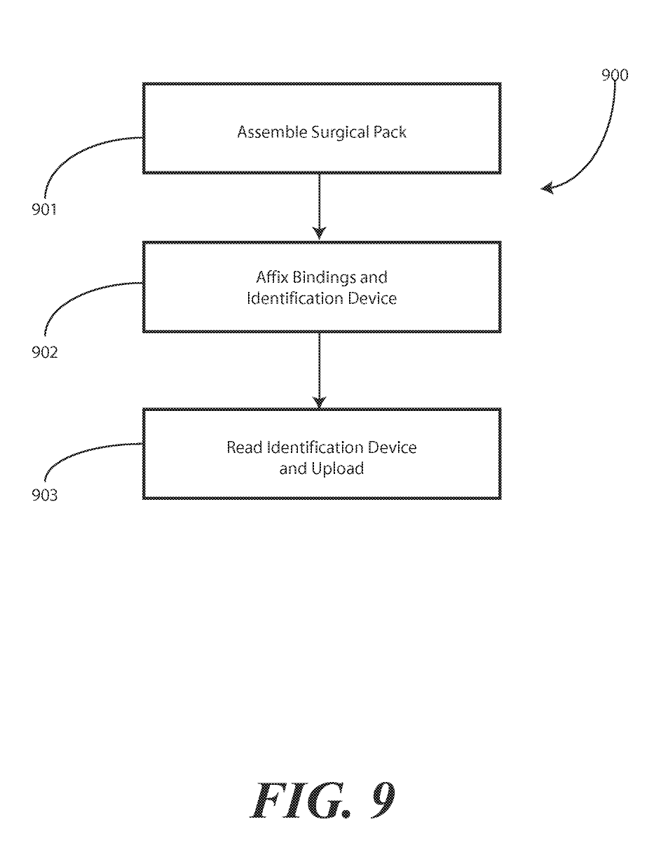

Turning now to FIG. 9, illustrated therein is a method 900 of making a surgical pack in accordance with embodiments of the invention. At step 901, the pack is assembled. As noted above, this can include assembling components that are later sterilized. Alternatively, it can include assembling sterilized components with non-sterilized components.

At step 902, adhesive bindings are attached to the completed back, with an identification device disposed between the bindings and the outer wrap. In one embodiment, the perforation of the identification device is disposed between the bindings such that when the bindings are removed, the identification device tears along the perforation, thereby rendering at least a portion of the identification device inoperable.

At step 903, the identity of the device is read by a remote reader disposed at the manufacturer's location. Information corresponding to the pack's identity can be uploaded into the manufacturer's computer system.

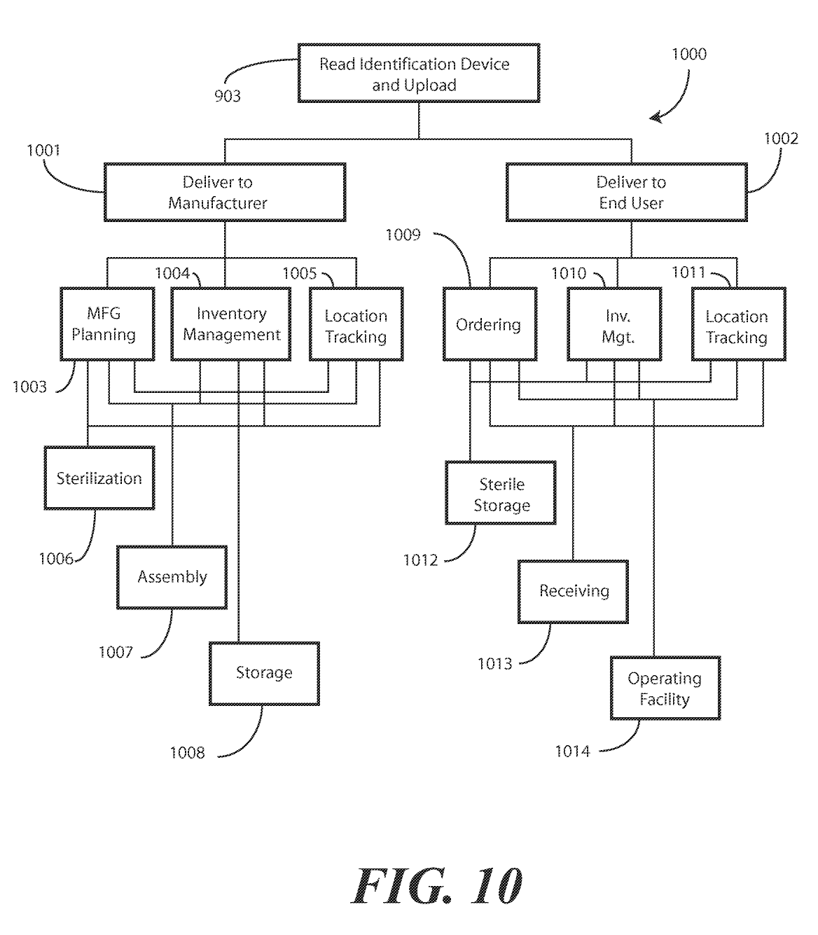

Turning now to FIG. 10, illustrated therein is a method 1000 for tracking the sealed pack in accordance with embodiments of the invention. The method 1000 begins where FIG. 9 ended, at step 903, in which the pack is read and uploaded into the system. This information can then be delivered to the manufacturer at step 1001. It can also be delivered to the end user at step 1002.

Once the information is delivered, it can be used in a variety of ways that have already been discussed above. Considering first the manufacturer's operations, these ways include manufacturing planning at step 1003. The information can be used for inventory management at step 1004. The information can also be used for location tracking at step 1005. As noted above, for one or more of these steps, this can include management in a variety of locations, including management at step 1006 in the sterilization location, management at step 1007 in the assembly location or manufacturing floor, and location at step 1008 in the storage facility.

Considering now the end user's operation, these ways include ordering planning at step 1009. The information can be used for inventory management at step 1010. The information can also be used for location tracking at step 1011. As noted above, for one or more of these steps, this can include management in a variety of locations, including management at step 1012 in the sterilized storage location, management at step 1013 in the receiving dock or other inventory storage locations, and location at step 1014 in the operating facilities.

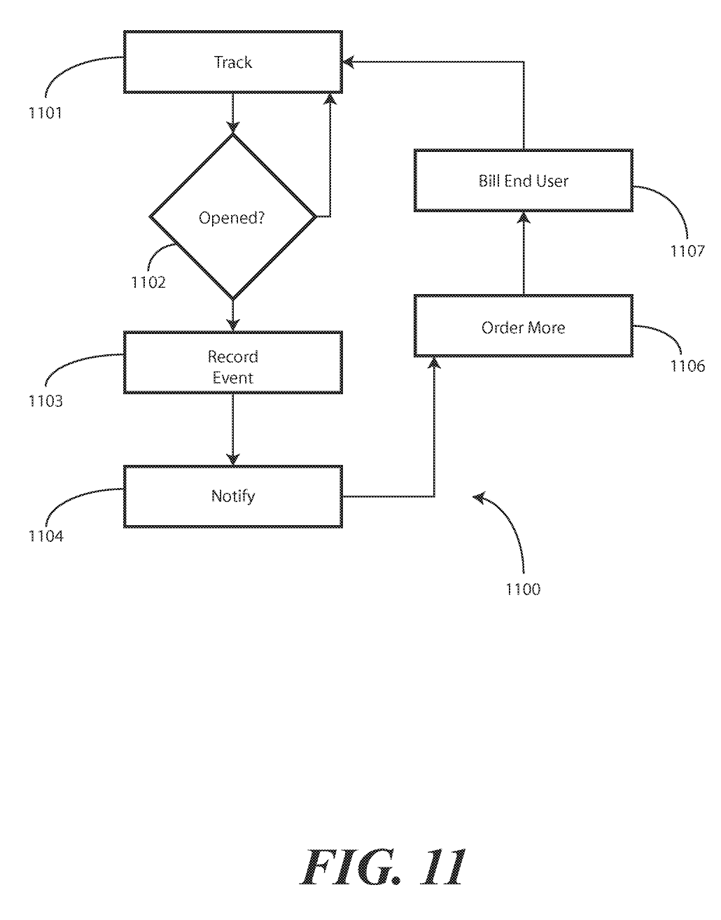

Turning now to FIG. 11, illustrated therein is a method 1100 for using the identification devices of embodiments of the present invention as described herein. At step 1101, the surgical packs to which the identification devices are attached can be tracked as previously described.

At decision 1102, remote readers can determine whether a pack has been opened. This is accomplished in one embodiment when the identification device is torn along its perforation, thereby changing the signals emitted by the identification pack that are readable by the remote readers. Other methods will be obvious to those of ordinary skill in the art having the benefit of this disclosure.

At step 1103, systems in accordance with embodiments of the present invention can record the opening event. At step 1104, the system can optionally notify one or both of the manufacturer and the end user. At optional step 1105, the system can bill either the end user or a third party, such as an insurance provider, that the pack has been used. At optional step 1106, replacement packs can be automatically ordered.

As described herein, identification devices can be used to identify when surgical packs have been opened. In one embodiment, this occurs when adhesive bindings are removed from outer wrap material, thereby tearing at least a portion of the identification device.

The identification devices can be read by remote readers, which in one embodiment are antenna based devices capable of interrogating each identification device with a radio-frequency signal. The read data is the delivered to computer systems at one or both of the manufacturer and end user for tracking and management. The information can be shared between the manufacturer and end user across a network.

In the foregoing specification, specific embodiments of the present invention have been described. However, one of ordinary skill in the art appreciates that various modifications and changes can be made without departing from the scope of the present invention as set forth in the claims below. Thus, while preferred embodiments of the invention have been illustrated and described, it is clear that the invention is not so limited. Numerous modifications, changes, variations, substitutions, and equivalents will occur to those skilled in the art without departing from the spirit and scope of the present invention as defined by the following claims. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims.

1. A method of assembling a surgical pack, the method comprising:
assembling contents of the surgical pack;
wrapping the surgical pack in an outer wrap;
affixing one or more adhesive bindings to the outer wrap, wherein an identification device is disposed between the one or more adhesive bindings and the outer wrap such that as long as the outer wrap is unopened, the identification device is capable of delivering a first identification signal to a remote reader.

2. The method of claim 1, wherein the identification device is configured such that when the outer wrap is opened, the identification device is physically deformed so as to deliver a second identification signal to the remote reader.

3. The method of claim 2, wherein the identification device is physically deformed by an act of tearing along a perforation.

4. The method of claim 3, wherein the identification device comprises a RFID device.

5. The method of claim 1, wherein the one or more adhesive bindings comprise a plurality of adhesive bindings.

6. A method of tracking a surgical pack, the method comprising:
detecting, by reading an identification device with a remote reader, whether the surgical pack has been opened; and
where the surgical pack has been opened, recording an opening event in a computer system operable with the remote reader.

7. The method of claim 6, further comprising ordering a replacement pack in response to the recording.

8. The method of claim 6, further comprising billing an end user in response to the recording.

9. The method of claim 6, further comprising notifying a manufacturer in response to the recording.

10. A method of inventory management for a plurality of surgical packs, with at least one of the plurality of surgical packs comprising contents different from at least another of the plurality of surgical packs, the method comprising:
identifying each of the plurality of surgical packs and the contents disposed therein;
detecting opening of at least one of the plurality of surgical packs; and
ordering a replacement pack having equivalent contents of the at least one of the plurality of surgical packs.

11. A method of detecting that a wrap layer disposed about a surgical pack has been unwrapped, the method comprising:
reading, with a remote reader operable with one or more processors, a first identification signal from an identification device coupled to the surgical pack;
reading, with the remote reader, a second identification signal from the identification device, wherein the first identification signal and the second identification signal are different and non-concurrent; and
recording an opening event with the one or more processors upon reading the second identification signal.
